(19) United States
(12) Patent Application Publication
(10) Pub. No.: US 2006/0263832 A1
(43) Pub. Date: Nov. 23, 2006

(54) Patterning of centrosomes and centrosome fragments as templates for directed growth of microtubules

(76) Inventors: Wen Shang, Clinton Park, NY (US); Jonathan S. Dordick, Schenectady, NY (US); Robert E. Palazzo, Latham, NY (US); Richard W. Siegel, Menands, NY (US)

Correspondence Address: ELMORE PATENT LAW GROUP, PC, 209 MAIN STREET, N. CHELMSFORD, MA, 01863, US

(21) Appl. No.: 11/435,156

(22) Filed: May 16, 2006

Related U.S. Application Data
(60) Provisional application No. 60/681,564, filed on May 16, 2005.

(51) Int. Cl. G01N 33/53; C12N 9/00; C12M 1/34

(52) U.S. Cl. 435/7.5; 435/183; 435/287.2

(57) ABSTRACT

The present invention relates to a new process to direct the growth and direction of polymerization of microtubules using patterned centrosomes or centrosome fragments on a surface. Incorporation a flow force to direct the position and the growth of microtubules, results in a regular network of microtubules. The invention therefore provides a new route to develop both sensing and non-sensing functional microtubule-based nanodevices such as those for nanoscale separation or purification.

### RELATED APPLICATION(S)

This application claims the benefit of U.S. Provisional Application No. 60/681,564, filed on May 16, 2005. The entire teachings of which are incorporated herein by reference.

### GOVERNMENT SUPPORT

The invention was supported by Lockheed-Martin Missiles and Fire Control-Orlando, the Nanoscale Science and Engineering Initiative of the National Science Foundation under NSF Award Number DMR-0117792, and National Institutes of Health Grant R01GM043264-12. The Government has certain rights in the invention.

### BACKGROUND OF THE INVENTION

In animal cells, centrosomes are the major microtubule organization centers responsible for nucleation, growth and orientation of microtubules. By organizing the microtubules, centrosomes play a critical role in controlling cytoskeletal architectures, thereby contributing to cell motility and function.

Microtubules are hollow 25-nm-diameter cylindrical protein biopolymers, built from tubulin, which form α and β tubulin heterodimers. They are one of the major components of the cytoskeleton in all eukaryotic cells and play important roles in the transportation of different vessicles and organelles within cells and in the segregation of chromosomes during cell division. On centrosomes, there are many small structures, known as γ tubulin ring complexes (γ-TuRC), (Gunawardane, R. N. et al., J. Cell Biol. 2000, 151, 1513; Vogel, J. M. et al., J. Cell Biol. 1997, 137, 193; Zheng, Y. et al., Nature, 1995, 378, 578) that serve as the nucleation sites for microtubules. When tubulin protein dimers are mixed with centrosomes, microtubules are able to be assembled onto centrosomes in the form of asters.

Microtubules grow from centrosomes with their minus ends embedded in the centrosomes and their plus ends extending away from the centrosomes. By organizing microtubules in this manner, centrosomes play an essential role in controlling cytoskeletal architectures, thereby contributing to cell motility and function.

Although scientists have long appreciated the important functions of centrosomes within cells, details about the molecular composition of centrosomes, their replication process, and how they nucleate and regulate microtubules still remain unclear. Studies involving centrosomes have expanded rapidly in recent years (Badano, J. L. et al., Nat. Rev. Genet. 2005, 6, 194; Ou, Y. and Rattner, J. B. Int. Rev. Cytol. 2004, 238, 119; Palazzo, R. E. and Schatten, G. P. Curr. Top. Dev. Biol. 2000, 49, 489; Suddith, A. W., et al., Methods Mol. Biol. 2001, 161, 215; Palazzo, R. E. and Davis, T. N. Methods Cell Biol. 2001, 67, 392; Ohta, T., et al., J. Cell Biol. 2002, 156, 87; Schnackenberg, B. J. and Palazzo, R. E. Methods Cell Biol. 2001, 67, 149), but so far no attempts to generate regular patterns of this unique organelle have been reported.

Because microtubules form the tracks along which microtubule-associated motor proteins can transport different cargos directionally, in recent years there has been a great interest to arrange microtubules into ordered arrays along which motor proteins and their cargo materials could be directionally moved on a molecular scale. Such efforts include immobilization of microtubules with antibodies complementary to the microtubules' minus ends; polymerization of long microtubules from short microtubule seeds with the growth of their minus end prevented by inhibitors; and alignment of microtubules onto a kinesin-coated surface in a microfluidic device. However, there have been no reports of attempts to apply centrosome-based templates for the control and alignment of microtubules.

Various techniques, such as microcontact printing (μCP) (Jiang, X. Y., et al., J. Am. Chem. Soc. 2003, 125, 2366-7; Chen, C. S., et al., Science 1997, 276, 1425-8) microfluidic channels (Chiu, D. T., et al., Proc. Nat. Acad. Sci. USA 2000, 97, 2408-13; Takayama, S., et al., Proc. Nat. Acad. Sci. USA 1999, 96, 5545-8) and elastomeric membranes (Ostuni, E., et al., Langmuir 2000, 16, 7811-9), have been developed to fabricate arrays of cellular or subcellular patterns. Although there are several approaches that have been demonstrated for the assembly of microtubules on surfaces (Limberis, L. et al., J. Nano Lett. 2001, 1, 277; Brown, T. B. and Hancock, W. O. Nano Lett. 2002, 2, 1131; Yokokawa, R. et al., Nano Lett. 2004, 4, 2265), the work on microtubules reported so far has focused on 1-D (one-dimensional) assembly. There have been no reports of building 2-D (two-dimensional) microtubule patterns with precise control of the positions and the growth directions.

Consequently, there remains a long-felt need for methods to achieve this goal as, arranging microtubules into ordered arrays is crucial to building microtubule-based nanodevices for directed movement of cargo materials and for directed assembly of different nanostructures.

### SUMMARY OF THE INVENTION

The present invention provides a new approach to pattern 2-D arrays of centrosomes and centrosome fragments. These patterns can be used as templates to control the position and growth of microtubules. Also disclosed is the discovery that when a liquid flow force is applied, the direction and polarity of the microtubules that ar polymerized from centrosome arrays can be controlled.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic representation of the setup used in the modified microcontact printing (μCp) process of the present invention. To improve the efficiency of the inking step, centrifugation was used to facilitate centrosome deposition. Further, a cone-shaped confining system was designed and fabricated for use in the centrifugation process.

FIG. 2 represents a schematic of the flow cell designed for use in alignment of the microtubules in directional and polarity studies.

### DETAILED DESCRIPTION OF THE INVENTION

A description of preferred embodiments of the invention follows.

The present invention provides a novel approach to pattern multi-dimensional arrays of centrosomes and centrosome fragments. These patterns are used as templates to control the position and growth of microtubules and, as such, have applications not only in the research and development of centrosome-based technologies, but also in the areas of nanobiology including molecular computers and microdevices, sensing and diagnostic technologies, separation and purification assays, force generation and drug delivery.

There are normally two approaches, direct and indirect patterning, for the fabrication of micro- or nanostructures using microcontact printing (μCp). In direct patterning, the patterned materials are directly transferred from polydimethylsilane (PDMS) stamps to surfaces, while in the indirect approach, a prepattern is made by microcontact printing and the materials that need to be patterned are placed on the top of the prepattern and selectively deposited onto the prepatterned locations. For patterning small biomolecules such as proteins, both approaches have been reported, while for patterning relatively large biomaterials, such as cells, indirect patterning is more frequently used (Krol, S., et al., A. Langmuir, 2005, 21, 705; Whitesides, G. M., et al., Annu. Rev. Biomed. Eng. 2001, 3, 335; Co, C. C. et al., J. Am. Chem. Soc. 2005, 127, 1598; Kane, R. S., et al., Biomaterials, 1999, 20, 2363).

In the present invention, considering the nature of the aqueous environment required for centrosomes to function, much effort was initially dedicated to the generation of centrosome arrays using the indirect approach. Different materials were patterned onto glass surfaces by microcontact printing with the purpose of immobilizing centrosomes selectively onto the desired areas through either physical or chemical interaction. The indirect process, however, proved to be inefficient because the high viscosity of sucrose in the centrosome solutions (typically 60% w/w) prevents the deposition of the centrosomes onto the prepatterns. The approach was also hampered by the relatively low concentration of centrosomes available in the solution making the deposition of a reasonable amount of centrosomes on the surfaces impossible. Since sucrose is essential for the isolation of centrosomes (Palazzo, R. E.; Vogel, J. M. Methods Cell Biol. 1999, 61, 35), and since the starting concentration of sucrose in the solution is so high, it is difficult to overcome the concentration effect in order to further increase the concentration of the centrosomes in the solution. The alternate approach of direct patterning by microcontact printing was therefore investigated to fabricate centrosome arrays. However, to directly pattern centrosomes, it became necessary to modify the microcontact printing process.

According to one aspect of the present invention, improved methods of microcontact printing are provided. Generally, microcontact printing involves three steps: inking the stamp with materials, drying the stamp, and then the printing step to transfer materials from the PDMS stamp to the desired substrate. In the conventional inking step, a solution of materials is placed on top of the PDMS stamp (drop-casting) and materials settle onto the stamp after a certain inking time. Again, even in the direct patterning approach, the high viscosity and relatively low concentration of centrosomes in solution made the deposition of a reasonable amount of centrosomes difficult. Simple drop-casting of the centrosome solution onto the PDMS stamp resulted in only a few centrosomes on the stamp after the inking step, even when the inking time was extended to 20 h. Centrosomes patterned using this process also lost their functionality during the long inking time.

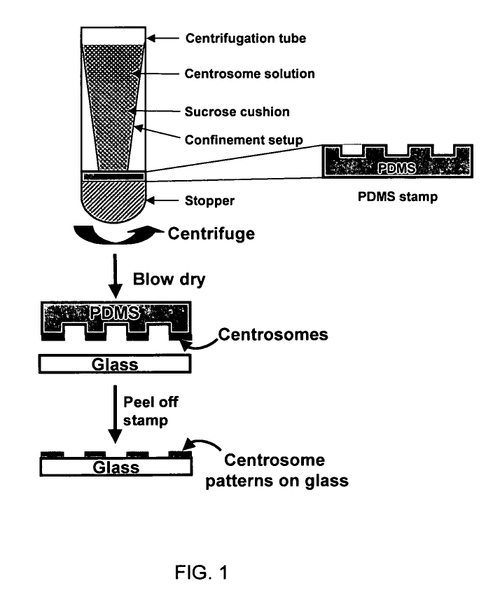

Therefore, in order to improve the centrosome deposition efficiency and to shorten the inking time, the inking step was modified. One improvement to this step involved the use of centrifugation to deposit the centrosomes onto the PDMS stamp. However, when using just a normal centrifuging setup (i.e., simply adding a centrifugation step), the area of the stamp exposed to the centrifuged solution was still too large to be covered with a reasonable amount of centrosomes for the printing process. To solve this problem, a cone shaped confining system for the centrifugation process was designed and fabricated (FIG. 1). After the above modifications (centrifugation and addition of the conical confinement system), the typical centrosome density on the stamp was increased 9 times and the inking time was reduced from 20 hours to merely 15 minutes. In order to keep the functionality of centrosomes, much care was also taken in the drying and printing steps. The PDMS stamp could only be blown dry briefly and the printing process had also to be done very quickly with a contact time of the stamp with the glass cover slip of only about 1-2 min.

As used herein, “centrosome” includes constituted centrosomes while “centrosome fragments” means the product of sonication or disruption of centrosomes or centrosome subcomponents produced by any means.

Using the above mentioned improvments and modifications centrosomes were successfully patterned into 2-D arrays. Advantages of these 2-D arrays include the ability to address and format each array individually with the ability to control spatial parameters and long range alignment of microtubules. These arrays may also be used as platforms to generate three dimensional (3-D) tracks with controlled polarity for controlled movement of materials in more than two dimensions.

In a further aspect of the invention, novel methods involving the use of centrosome fragments as arrayed templates for microtubule polymerization are provided. The present invention demonstrates that both centrosomes and centrosome fragments formed templates that retained their microtubule organization function. The positions of microtubules that grew from the templates were well defined by the templates. Compared to fully constituted centrosomes, the patterning of centrosome fragments is simpler and more straightforward, and thus should broaden the application of centrosome-based materials in the assembly of microtubules, as well as the construction of microtubule-related micro- or nanoscale devices.

As used herein, an “array” is any arrangment of a set of points. As used herein an array can be produced from any microtubule organization center including, but not limited to, centrosomes, centrosome fragments, expressed centrosome proteins, gamma-tubulin ring complexes or microtubule nucleation seeds. When formed on a substrate, the array generated from a microtubule organization center is termed a “template.” Further, the points of an array may be used to produce templates for the ordered and/or directed growth of microtubules.

As used herein “patterned or ordered arrays” refer to the non-random, pre-determined spacing of one or more microtubule organization centers into lines, circles (radial lines), blocks, sites, sections or zones. The spacing of line widths and between ordered or patterned sites in any array will be determined, in part, by the size of the microtubule organization center (centrosome or centrosome fragment) size. The spacing for a centrosome array may be from 2 μm to 1000 μm with a preferred range of between 2 μm to 10 μm. For centrosome fragment arrays the width and spacing can be from between 25 nm to 1000 μm with a preferred range from 25 μm to about 10 μm.

Further to the development of arrays of centrosomes and centrosome fragments as microtubule organization centers, the present invention provides methods of controlling the growth, direction of growth and polarity of microtubules using flow force and capillary force inside microfluidic channels or MMIC (micromolding in capillaries). This approach provides a unique platform to generate different microtubule patterns and to control the orientation of microtubules by different fields. These different fields include, but are not limited to, those such as liquid-flow field, electric field, and magnetic field and the like.

As used herein, “microtubule growth” means the polymerization of tubulin into microtubule polymers. “Directed growth of microtubules” means microtubule growth patterned on a template of centrosomes or centrosome fragments.

In one embodiment of the invention, arrays of centrosomes and centrosome fragments can be used to study different behaviors of centrosomes alone or as in cellular development under different physiologic or laboratory conditions. As such, these arrays are used in centrosome-related drug screening and in the research and development of centrosome and microtubule biology.

In recent years, the fields of biology and nanotechnology have converged and a new field, nanobiotechnology, has emerged. This new area involves fundamental studies of the interaction between biological materials and nanoscale surfaces, (Labarre, D. et al., Biomaterials, 2005, 26, 5075; Vertegel, A. A et al., Langmuir, 2004, 20, 6800; Gun'ko, V. M., et al., J. Colloid Interface Sci., 2003, 260, 56; Monteiro-Riviere, N. A., et al., Toxicol. Lett. 2005, 155, 377), exploration of methodologies to synthesize biologically based hybrid materials (Singh, R., et al., J. Am. Chem. Soc., 2005, 127, 4388; Krol, S., et al., Langmuir, 2005, 21, 705; Guo, Z., et al., Adv. Mater. 1998, 10, 701; Tsang, S. C., et al., J. Chem. Soc., Chem. Commun. 1995, 2579; Lenihan, J. S., et al., J. Nanosci. Nanotechnol. 2004, 4, 600; Jiang, K., et al., J. Mater. Chem. 2004, 14, 37), and the design and fabrication of functional bio-based nanodevices (Prokop, A., et al., J. Biotechnol. Bioeng. 2002, 78, 459; Djalali, R., et al., Polym. Mater. Sci. Eng. 2003, 89, 273; Cao, Y. C., et al., J. Am. Chem. Soc. 2003, 125, 14676; Chen, R. J., et al., J. Am. Chem. Soc. 2001, 123, 3838; Chen, R. J., et al., Proc. Natl. Acad. Sci. U.S. A. 2003, 100, 4984; Kuenzi, P. A., et al., Microelectron. Eng. 2005, 78-79, 582; Meiring, J. E., et al., Chem. Mater. 2004, 16, 5574; Braun, E. and Keren, K. Adv. Phys. 2004, 53, 441; Liu, H., et al., Nature Mater. 2002, 1, 173; Bianco, A. and Prato, M. Adv. Mater. 2003, 15, 1765). In building functional biomaterials based nanodevices, one critical issue is to assemble biomaterials into well-defined locations at the micro- or nanoscale.

One embodiment of the invention provides a process to assemble a subcellular structure, centrosomes, into ordered 2-D arrays using a modified microcontact printing process. Assembly of biologically-based materials into ordered structures at micro- and nanoscale is the key for building functional nanobiological devices. The advantages to use centrosome fragments include scaling down the pattern size to the nanoscale and alternating the density of microtubules that grow from centrosome-fragment templates. In the present invention, it is discovered that small centrosome fragments possess microtubule nucleating capability. The use of this new material to assemble microtubules on glass surfaces has also demonstrated. The invention contemplates the use of other surfaces for template assembly of microtubules including, but not limited to, plastics, metals, polymers including biological substrates and artificial surfaces, silicon, and other substrates such as for example, ceramic substrates and/or hybrid/composite substrates. Assembly on such substrates by this method is superior to other methods such as those based on proximal probes, e.g., STM (Scanning Tunneling Microscopy) or AFM (Atomic Force Microscopy). The substrates used herein may be of different shapes and volumes. These include, but are not limited to, flat/planar, circular, square, triangular, oval, torroidal, or any three dimensional volume having substantially the said shape. As such, the arrays and/or templates deposited on said shapes may also take on the same shape or occupy the same dimensions in space.

For microtubules organized by patterned centrosomes/fragments, motor protein, dyneins, can be used to assemble materials on the patterned centrosome/fragment arrays. It will also facilitate the assembly of different materials at the same spots or locations on the arrays. The present invention also contemplates the use of “caged” ATP for controlled movement of motor proteins.

In one aspect of the invention, the arrayed templates can be used in the development of molecular computers and related biodevices. Combining the patterning of the isolated centrosomes and the directed growth of microtubules disclosed herein will lead to the generation of a desired microtubule network which can then be co-opted or designed to transmit signals used in molecular computing devices. The read-out of a molecular computer by a molecular shuttle, here microtubule-associated molecular proteins, could solve the current problem associated with interconnects of these devices.

The microtubule tracks created using the present invention may also be used in the construction of intelligent microtubule-related molecular machines (Bachand, G. D.; Rivera, S. B., et al., Nano Lett. 2004, 4, 817-821; Bohm, K. J. et al., Nanotechnology 2001, 12, 238-244; Hess, H. and Vogel, V. Rev. Mol. Biotechnol. 2001, 82, 67-85; Kinbara, K. and Aida, T. Chem. Rev. 2005, 105, 1377-1400; Limberis, L. and Stewart, R. J. Nanotechnology 2000, 11, 47-51). Furthermore, such microtubule tracks may then be used to manipulate filaments or polymers such as DNA in natural and artificial biologic systems.

The arrays of the present invention have applications and may be useful in sensing, diagnostic and in separations and purifications technologies. Modification or functionalization of these ordered microtubules with biological or nonbiological materials could also offer a platform for the development of novel, highly sensitive and selective devices to meet a variety of sensing and diagnostic needs. Understanding and controlling the growth of microtubules are crucial for constructing microtubule-based sensors and device components. Further modification or functionalization of these ordered microtubules will offer a biologically-based platform for biological and chemical sensors. These organized arrays are ideally suited for further biomolecular and material hybrid functionalization over a sufficiently wide range of length scales to accommodate a variety of sensing needs. Such functionalization includes attachment of proteins with natural affinity for microtubules (e.g., kinesin), proteins with no known affinity for microtubules, but with specialized properties (e.g., enzymes and antibodies that can be chemically attached), material tags (e.g., fluorophores, metal ions, magnetic nanoparticles), and other biological and nonbiological materials.

In one embodiment, the present invention contemplates the use of these arrayed templates with kinesin functionalized with simple metal oxide nanoparticles, which may endow the microtubules with the capability to sense into the infrared and microwave radiation regimes (e.g., biologically-inspired antennae). For example, functionalization of microtubules with kinesin, that can move unidirectionally along the microtubules, can be used to transport fluorescent tags (e.g., quantum dots or fluorophore labels) along microtubule chains. Chemical functionalization is also contemplated. Using enzymes and antibodies to demonstrate the functional properties of these proteins via enzymatic assays and antibody binding studies are also contemplated. Enzymatic assays and antibody binding assays are well known in the art. It is also contemplated that microtubules may be, themselves, used as templates for functionalization with nonbiological hybrid materials, including metal or metal oxide nanoparticles. Functionalization of any of the compontents of the system of the present invention is further contemplated. This functionalization may occur before, during or after microtubule polymerization and may include the addition of moities to the substrate, templates, tubulin monomers, or additions which occur by the addition of a solvent or wash.

In one embodiment of the invention, materials to be sensed are selected and/or collected or directionally deposited to one position or site on an array for analysis. The invention allows testing of material concentrations or signals even at very low concentration. These platforms will enable the development of novel, highly sensitive and selective devices over a range of length and matrix scales to meet a variety of sensing and actuating needs.

In another embodiment of the invention, the sensing signals are movable on the microtubule-formed tracks. A moving signal is much easier to detect than a stationary signal. So from this point of view, it can sense very low concentration of materials. It is also recoverable and reusable.

In a further embodiment, antibodies may be placed or sited on the centrosome or centrosome fragment spots of the arrayed templates. In this case, kinesin or other carrier/functionalized proteins carry an antigen along a microtubule track producing signals at individual or predetermined sites or spots for further analysis. Modification or functionalization of these ordered microtubules with biological or nonbiological materials will offer a platform for the development of novel, highly sensitive and selective devices to meet a variety of sensing and actuating needs. Using ordered microtubule arrays combined with kinesin or dyneins, analytes can be colleted and/or concentrated into one position and then detected at the destination spot. In this case, the velocity or movement of the analyte would not need to be detected, only the static signal (IR, Raman, or other signals) at the detection station.

In one embodiment of the invention, the ordered polymerization of microtubules are used to generate motive forces. Single motor protein molecules can generate a small force. On an array of aligned and polarized microtubules, large numbers of motor proteins could produce relatively large, directed, cumulative forces. Such force could be used to power pumps or valves in fluidic devices over a wide range of length scale. These forces can also be exploited in implantable medical devices, for example, for controlled drug delivery. Such medical devices include any device that may be implanted into a subject's body including, but not limited to, implantable drug pumps and inserts, neurological stimulators, cochlear devices and the like.

The microtubule arrays and applications of the arrays of the present invention have a range of applications in the area of drug delivery. As such, formation of microtubule lattices in multidimensions can be used to facilitate wound healing by providing a framework or carrier system on which thereapeutics or natural biological molecules can be transported. For example, preformed arrays may be used to facilitate healing after surgery or injury and may take the form of implanted devices, coatings for implants, stents, or on transplanted tissues or organs.

Materials

The following chemicals and proteins were used as received: PIPES (1,4-piperazinebis(ethanesulfonic acid)), EGTA (ethylene glycol-bis(2-aminoethyl-ether)N,N,N′,N′-tetraacetic acid), GTP (guanosine 5′-triphosphate sodium salt); magnesium sulfate and sodium borohydride from Fisher Scientific (Morris Plains, N.J.); glutaraldehyde (25% in aqueous solution, EM grad) from Electron Microscopy Sciences (Fort Washington, Pa.); Sylgard 184 from Dow Coming (Midland, Mich.); 3-aminopropyltriethoxysilane (APTES), phosphate-buffered saline tablets, and mouse anti-gamma tubulin antibody from Sigma (St. Louis, Mo.); glutaraldehyde (8% in aqueous solution, EM grad) from Electron Microscopy Sciences (Fort Washington, Pa.); Sylgard 184 from Dow Coming (Midland, Mich.); Texas Red affinity purified Goat Anti-Mouse IgG (H+L) from Jackson ImmunoResearch Laboratories, Inc. (West Grove, Pa.); Rat anti-alpha tubulin from Serotec (Raleigh, N.C.); Paclitaxel and goat anti-rat Alexa Fluor 488 from Molecular Probes (Eugene, Oreg.).

The phosphate-buffered saline tablets were dissolved in Nanopure water and diluted to the desired concentration (PBS buffer, 137 mM NaCl, 2.7 mM KCl, and 10 mM phosphate buffer, pH=7.4). The PEM buffer solution was made with 5 mM Pipes, 1 mM EGTA, and 1 mM MgSO₄ and was adjusted to pH 7.0 with KOH. The reassembly buffer contains 100 mM Pipes, 1 mM EGTA, 5 mM MgSO₄, and 2 mM GTP and its pH was adjusted to 6.9 with KOH. The substrates for all the samples are round glass cover slips were (12-mm in diameter) purchased from BellCo Glass (Vineland, N.J.). Before each use, these cover slips were immersed in concentrated H₂SO₄ for several hours followed by multiple washes with Nanopure water and blow dried with a stream of air or nitrogen.

Centrosomes

Centrosomes were isolated from frozen-stored Spisula oocyte lysates using sucrose density-gradient fraction methods previously described (Vogel, J. M., et al., J. Cell Biol. 1997, 137, 193-202). All the centrosome samples were stored at −80° C. for further use. Sea urchin (Strongylocentrotus purpuratus) microtubule protein was prepared by three cycles of polymerization/depolymerization as previously described. (Vogel, J. M., et. al., J. Cell Biol. 1997, 137, 193-202; Suprenant, K. A., and Marsh, J. C. J. Cell Sci. 1987, 87, 71-84; Mitchison, T. J. and Kirschner, M. W. Methods Enzymol. 1986, 134, 261; Palazzo, R. E.; Vogel, J. M. Methods Cell Biol. 1999, 61, 35). The tubulin samples were diluted with reassembly buffer to 0.7 mg/ml (or 2 mg/ml for the modified microcontacting method) determined by protein assay and stored at −80° C. in small aliquots.

Fabrication of PDMS Stamps

Details of the PDMS (poly dimethysiloxane) stamp fabrication process can be found in the review by Xia and Whitesides (Xia, Y.; Whitesides, G. M. Angew. Chem. Int. Ed. 1998, 37, 550). Basically, a master pattern, mostly generated by photolithography, was passivated using fluorosilane. Sylgard 184 was well mixed (A:B ratio ˜1:10) and degassed for ˜20 min in vacuum. The mixed precursor was then cast against the master pattern and degassed again for ˜1 hour. After being baked at 60° C. for one hour, the precursor was cured and the PDMS stamp was thus generated by peeling off the cured polymer from the master.

Immunofluorescence

Centrosomes, centrosome fragments and microtubules were visualized by immunofluorescence as previously described (Mitchison, T. J., and Kirschner, M. Methods Enzymol. 1986, 134, 261-268; Palazzo, R. E., Vaisberg, E., Cole, R. W., and Rieder, C. L. Science 1992, 256, 219-221). Samples were first washed with PEM buffer and then fixed by adding 50 μL of 2% glutaraldehyde in reassembly buffer for 15-20 min at room temperature. Samples were then postfixed in −20° C. methanol for 1-12 h. After fixing with glutaraldehyde, samples were washed with PBS, and treated with 10 mg/ml sodium boronhydride. After blocking with 5% nonfat dry milk in PBS for 20 min, samples were incubated in primary antibody, mouse anti-gamma tubulin to label centrosomes or rat anti-alpha tubulin to label microtubules, for 15 min. each. For the modified microcontact printing technique, the incubation time was 20 min. Samples were then washed with PBS three-times and incubated in secondary antibody, rhodamine-conjugated goat anti-mouse, for an additional 15 min. For the modified microcontact printing method, Texas Red AffiniPure goat anti-mouse IgG (H+L) and goat anti-rat Alexa Fluoro 488 were used with incubation lasting 20 min. Samples were then washed with PBS three times and permanently mounted. All images of the samples were taken with a Zeiss Axioplan epifluorescence microscope equipped with a 40×/1.3 NA, 63×/1.3 NA or 100×/1.3 NA objective lens and a Hamamatsu SIT-video camera or a Coolsnap HQ camera (Photometrics) that was coupled to a Metamorph image processing system (Version 6.2r4, Universal Imaging, Media, Pa.).

The invention will be further described with reference to the following examples; however, it is to be understood that the invention is not limited to such examples.

### EXAMPLES

### Example 1

### Deposition of Centrosomes Using Microcontact Printing (μCp)

Microcontact printing is a soft lithographic method that has been widely used for protein and cell patterning. Using this method, a clean polydimethysiloxane (PDMS) stamp with micron-scale features (posts or lines) was covered with 10 μL of 10⁷/mL centrosome solution and inked for 20 h. The stamp was placed in a petri dish with a filter paper moistened by Nanopure water at the bottom to prevent the drying of centrosome solution during the inking process. The stamp was then rinsed with PBS buffer, blow dried, and then brought into contact with a clean cover slip. The PDMS stamp was peeled off from the substrate after about an hour of printing.

Using this drop-cast method, centrosomes were successfully transferred from the PDMS stamp to the desired area. There were also background patterns of lines or dots that corresponded to original patterns in the PDMS stamp. The contrast in these background patterns appeared to be due to other protein components, perhaps contaminants present in the original centrosome samples. During microcontact printing, these proteins were also transferred and thus generated background patterns. The centrosome density on the surface, however, was not sufficient to form continuous patterns. It was hypothesized that this was may be due to low centrosome concentration arising from the relatively low starting concentration of the samples or as a product of the viscosity of the sucrose preparation which results in less diffusion of centrosomes to the stamp. The highest concentration of centrosome samples currently isolated is approximately 10⁷/mL. Typically a 5 μL sample was inked in a 4-mm² area on the PDMS stamp. This resulted in about 5×10⁴ centrosomes on the inked areas. A centrosome is approximately 2 μm in dimension. Assuming centrosomes are square, approximately 5×10⁴ centrosomes will form a monolayer of about 0.2 mm². Even with 100% deposition efficiency, the surface coverage will only be about 5% and centrosomes will not form continuous lines. Secondly, there is a high concentration of sucrose (about 60%) in the centrosome solution. Consequently, the viscosity of the solution is also high, so only a small portion of the centrosomes may be able to diffuse through the sucrose and be absorbed onto the stamp surface during the duration of the inking process.

In an effort to resolve this issue, centrifugation during the inking process was used in the hope of depositing more of the organelles onto the stamp. Unfortunately, the background contrast caused by the other proteins printed together with the centrosomes was too low to enable identification of ordered arrays.

### Example 2

### Assembly of Centrosomes Using Patterned Substrates

Substrates having different surface modifications are important in building interfacial devices. To this end, studies of directed assembly of centrosomes using patterned substrates were undertaken. To guide the assembly of centrosomes, patterns of 3-aminopropyltriethoxysilane (APTES) were first fabricated on glass cover slips using micromolding in capillary (MIMIC).

Micromolding in a capillary is another soft-lithographic method that is used for the patterning of interconnected micro- and nanostructures. During the patterning process, the PDMS stamp is placed on the surface of the substrate and a network of channels form between the stamp and the substrate. When a solution is placed at the open ends of these channels, it spontaneously fills the channels by capillary force, if the solvent in the solution wets the PDMS surface.

Following this rationale, a clean PDMS stamp with patterns of lines was placed on the surface of a cover slip and formed conformal contact with the glass surface. Both ends of line patterns were cut open using a razor blade. A small drop of 2% (v/v) APTES in 95% (v/v) ethanol/5% H₂O (adjusted with acetic acid to pH 5) was placed on one side of the channels. Ethanol wetted the PDMS and the solution filled the channel fairly rapidly with the relatively low viscosity ethanol solution. After exposing the cover slip to air for several hours, ethanol was completely evaporated and the PDMS stamp was separated from the substrate. The substrate was then rinsed with ethanol thoroughly, dried, and baked at 120° C. on a hot plate for 30 min. Patterns of APTES lines were formed on the cover slips.

A drop of centrosome solution was then placed on the patterned cover slips for 5-10 h. Again, due to the high concentration of sucrose in centrosome samples, and corresponding high viscosity of the solution, the efficiency of the adsorption process was low. Simply applying centrosome solution onto the APTES patterned surfaces only provided a few centrosomes for each sample, even after one day.

In order to improve the density of centrosomes in the final pattern, a centrifugation step during the desposition process was added. During centrifugation, cover slips were placed into the bottom of centrifuge tubes with the APTES patterns facing up. This was followed by adding 2 mL of 10% sucrose solution in PEM buffer into the tube. Centrosome solution (1 mL) was then added carefully onto the top of the sucrose solution. The centrosomes were then brought to the cover slip by centrifugation using a centrifuge rotor (model JS 13.1; Beckman Instruments) at 10,000× g and 4° C. for 15 min. In this pattern-guided assembly of centrosomes, centrifuging deposited more centrosomes onto the substrate than the simple drop-cast process. Under fluorescence microscopy, both APTES patterns and centrosomes are seen. Most centrosomes selectively absorbed onto the patterned APTES lines. They still did not form continuous line patterns, even with centrifugation. Further, some nonspecific adsorption of centrosomes did occur in the nonpatterned areas. The selectivity of centrosomes on APTES surface to glass surface is about 3:1. Nevertheless, the high ratio of centrosomes in the pattern versus those randomly distributed is sufficient for further studies involving microtubule orientation.

The results demonstrated in Examples 1 and 2 represent the first time that centrosomes have been patterned by either method into ordered arrays. Despite the low concentration of deposition, both routes still produce ordered centrosome arrays which may be used in the construction of future sensor platform and other applications detailed herein.

### Example 3

### Guided Growth of Microtubules

Inspired by the capillary flow to pattern APTES onto a substrate, fluid flow alignment of microtubules in microchannels has been accomplished. The strong capillary force inside the PDMS microchannles indeed aligned the microtubules within these channels. To control the orientation of the microtubules in one dimension (1-D), an approach similar to micromolding in the capillary (MIMIC) was applied. Here the MIMIC process is not only being used to spatially position the microtubules, but also using a flow force generated inside the microchannels to guide the growth of microtubules. The PDMS stamp was treated with plasma cleaner for one minute prior to sample preparation in order to change its surface from hydrophobic to hydrophilic. The aqueous solution of tubulin can thus enter the microchannels formed between the PDMS stamp and the glass substrates. To control the growth of microtubules, the tubulin solution (0.7 mg/mL) was first thawed on ice, followed by polymerization of the tubulin monomers at room temperature for 30 min. The partially polymerized tubulin solution was then placed on one side of the open-ended microchannels. The substrate was placed in a petri dish with a filter paper moistened by Nanopure water at the bottom to prevent the evaporation of tubulin solution. Samples were left at room temperature for 45 min. A 2% (w/v) glutaraldehyde solution in reassembly buffer was then placed at the same end of the microchannels to fix the microtubules and also further align microtubules along the flow direction. After complete evaporation of the solvent, the samples were processed for immunofluorescence analysis in order to visualize the aligned microtubules. As a result, ordered 1-D microtubule patterns were generated within channels with widths of 12 μm and 2 μm spacing. Consequently, using a fluid flow force inside the microchannels provides a reliable approach for generating ordered arrays of microtubules in 1-D.

Compared to research from other groups on aligning microtubules (Samira G. M., et al., Nano Lett. 2003, 3, 633-637; Brown, T. B., and Hancock, W. O. Nano Lett. 2002, 2, 1131-1135; Dennis, J. R., et al., Nanotechnology 1999, 10, 232-236; Hess, H., et al., Nano Lett. 2001, 1, 235-239), this method has several advantages: 1) it can provide ordered microtubules with tunable spacing; 2) it can control the growth of microtubules at the microscale; and 3) depending on the size of the channels defined by the PDMS stamps, either microtubule bundles or microtubule wires can be generated. This demonstration provides a useful set of tools for constructing ordered microtubule networks using ordered centrosome arrays to initiate microtubule polymerization and flow to orient microtubules originating from those centers.

### Example 4

### Fabrication of 2-D Arrays of Centrosomes by Modified Microcontact Printing (μCp)

As one of the most extensively used soft lithographic methods, microcontact printing has been applied to a wide range of fields that involve micro- and nano-fabrications (Whitesides, G. M., et al., Annu. Rev. Biomed. Eng. 2001, 3, 335; Xia, Y. and Whitesides, G. M. Angew. Chem. Int. Ed. 1998, 37, 550) and much work has been done in patterning proteins and cells on surfaces using microcontact printing (Krol, S., et al., Langmuir, 2005, 21, 705; Whitesides, G. M., et al., Annu. Rev. Biomed. Eng. 2001, 3, 335; Co, C. C., et al., J. Am. Chem. Soc. 2005, 127, 1598; Bernard, A., et al., Adv. Mater. 2000, 12, 1067; Schmalenberg, K. E., et al., Biomaterials, 2004, 25, 1851; Kane, R. S., et al., Biomaterials, 1999, 20, 2363).

To pattern centrosomes in the least intrusive fashion, a microcontact printing (μCp) process was modified and improved for the patterning of 2-D centrosome arrays. The improvement to produce the microcontact printing process of the present invention results in the successful patterning of centrosomes with microtubule organization function remaining intact. Using this method, microtubules were grown from the desired locations defined by the centrosome template.

Centrosomes were patterned on a glass cover slip by microcontact printing as described above with the following modifications and improvements. The setup for the modified process is illustrated in FIG. 1.

A clean polydimethysiloxane (PDMS) stamp with patterns of 10 μm posts separated by 10 μm was placed in the bottom of a centrifuge tube with the patterns facing up. The setup further contains a PDMS stopper. A plastic pippet tip was then placed on the top of the stamp and tightened in order to confine the contact area of the solution with the stamp. This was followed by adding 2 mL of 10% sucrose solution in PEM buffer into the tube. A diluted centrosome solution (1 mL, 0.4 million/mL) was then added carefully onto the top of the sucrose solution. The centrosomes were then brought to the PDMS stamp by centrifugation using a centrifuge rotor (model JS 13.1; Beckman Instruments) at 10,000×g and 4° C. for 15 min. The stamp was then taken out of the tube, briefly dried, and immediately brought into contact with a clean cover slip. To clean coverslips before patterning, cover slips were immersed in concentrated H₂SO₄ for several hours followed by multiple washes with Nanopure water and ethanol. They were then blow dried with a stream of nitrogen. The PDMS stamp was peeled off from the substrate after one minute of printing. Immunofluoresence of the resulting arrays, revealed that centrosomes were successfully transferred from the PDMS stamp to the desired areas. There were background patterns of dots that corresponded to original patterns in the PDMS stamp. Contrast in these background patterns appeared to be due to other cell components present in the original centrosome solutions. During conventional microcontact printing, these components were also transferred and thus generated background patterns. Most centrosomes on the patterns were singles with sizes around 2 μm, while some were doubles, and some formed aggregates.

Therefore, the improvements described herein result in ordered arrays of centrosomes of sufficient quantity and signal over background to be useful in the applications detailed herein.

### Example 5

### Directed Growth of Microtubules from Patterned Centrosomes

After the centrosomes were patterned into ordered arrays using the modified microcontact printing process of Example 4, they were used as a template for the directed growth of microtubules. By applying a liquid flow force to the patterned arrays the growth direction and polarity of the microtubules were also controlled.

Sea urchin tubulin was first thawed on ice, followed by dilution to 0.5 mg/mg with reassembly buffer. Fifty μL of tubulin solution was placed on the patterned centrosome surface as soon as the PDMS stamp was peeled off and allowed to grow at a temperature of 26° C. for 30 min. During the tubulin polymerization process, the substrate was placed in a petri dish with a filter paper moistened by Nanopure water at the bottom to prevent the evaporation of tubulin solution. Samples were then fixed by 50 μL of 2% glutaraldehyde in reassembly buffer for 15-20 min at room temperature and were processed for further immunofluorescence assay.

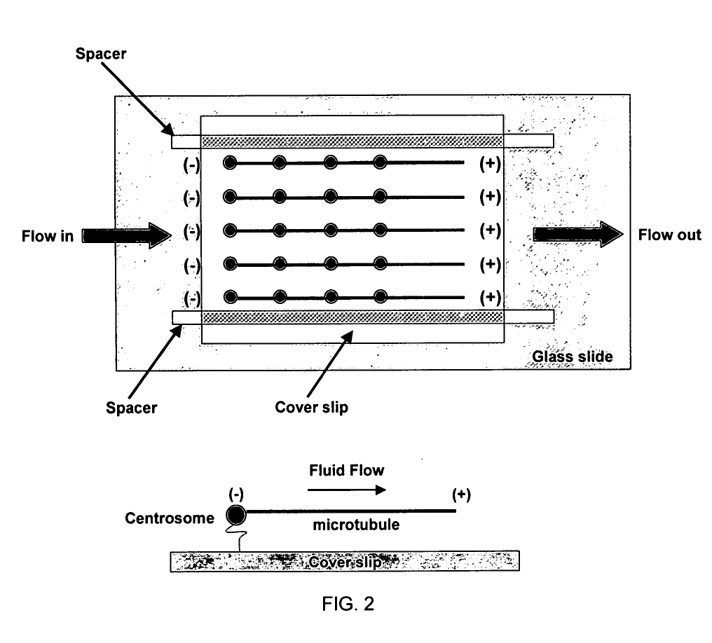

A flow cell, as illustrated in FIG. 2, was set up in order to align microtubules that grow from patterned centrosomes in a desired direction. A glass slide (3 in×3 in×1 mm) was cleaned with Nanopure water and ethanol first then two PDMS spacers were placed on the slide that formed a macrochannel with a width of 2 mm and height of ˜200 μm. After patterning centrosomes on a cover slip (˜1 cm×1 cm×0.12 mm), the cover slip was immediately put on top of the PDMS spacers and a flow cell was formed. The tubulin solution was flowed into the cell as soon as the cell was set up and microtubules were allowed to grow at room temperature for 15 minutes. After a wash with 200 μL of 20 μM paclitaxel solution and with 400 μL reassembly buffer by flowing the solution inside the cell, 50 μL more tubulin solution was flowed into the cell and allowed to grow for another 10 minutes at room temperature. A 200 μL 2% (w/v) glutaraldehyde solution was then flowed through the channel in order to fix the centrosomes and microtubules and also to further align the microtubules along the flow direction. After 15 min, the cover slip was detached from the slide and was washed three times with PBS buffer and then processed for further immunofluorescence analysis.

The results of immunofluorescence revealed that microtubules grew from almost all of the patterned centrosomes after being incubated for 30 min at 26° C. Using this approach, the positions of the microtubule asters could be controlled on the substrate.

### Example 6

### Control of Microtubule Polarity

By applying a liquid flow force to the patterned arrays developed in Examples 4 and 5, not only can the growth direction of the microtubules be controlled but the polarity of the microtubules can also be controlled.

While the polarity of the aligned microtubules may be tested by a microtubule-associated protein, kinesin, which is a plus-end directed motor, here it was not necesssary to do so since the minus-end is always on the centrosome side and the plus end is downstream, along the flow field. It was expected that the polarity of the polymers will be bipolar, with random alternating polymers polarized in opposite directions. The intrinsic structural polarity of microtubules comes from the fact that all the tubulin dimers that constitute the microtubules stack together in the same fashion along the cylindrical walls. In animal cells, microtubules are essential to many directional movements of organelles and other cell components. In recent years, there has been great interest to arrange microtubules into ordered “tracks” along which different cargo materials could be unidirectionally transported by microtubule-associated motor proteins. There are several different approaches that have been reported for the alignment of microtubules and the control of their polarity. These include immobilization of microtubules with antibodies complementary to microtubules' minus ends (Limberis, L., et al., J. Nano Lett. 2001, 1, 277); polymerization of long microtubules from short microtubule seeds, with the growth of their minus end prevented by inhibitors (Brown, T. B. and Hancock, W. O. Nano Lett. 2002, 2, 1131); and orientation of microtubules on a kinesin-coated surface in a microfluidic device (Yokokawa, R et al., Nano Lett. 2004, 4, 2265).

A flow set up (as shown in FIG. 2) was successfully applied to align the direction of microtubules that grew from patterned centrosomes. All microtubules have their minus ends capped in the centrosomes and plus ends extend away from the centrosomes to form asters, if left unperturbed. When a liquid flow field was applied during and after the growth of the microtubules from the centrosome template, the polarity of the microtubules was aligned by the flow field with the microtubule minus ends pointing upstream and the plus ends pointing downstream. Centrosomes were patterned by microcontact printing as described herein and microtubules were not only grown from the patterned centrosomes, but also oriented along the flow force direction. This capability for microtubule alignment promises a new method for creating one-dimensional routes for microtubule-associated motor proteins and the cargos that they carry to move unidirectionally. Furthermore, building multidimensional arrays may now be possible.

The studies using the modified microcontact printing technique disclosed herein resulting in the directed growth and polarity of microtubules demonstrate that centrosomes patterned by the modified microcontact printing technique maintained their microtubule organization function. The growth of microtubules was directed by the patterned centrosome template. Using a flow field, both the direction and the polarity of the microtubules could be controlled. These results promise a potentially useful new tool for constructing microtubule networks using ordered centrosome arrays. A 2-D array based upon centrosomes and microtubules could likely be assembled for the unidirectional movement of different cargo materials carried by microtubule motor proteins. When microtubules grew from randomly distributed centrosomes, they were organized by those centrosomes, but without any precise control of their positions on surface. When microtubules grew from ordered centrosomes however, they were not only organized by these ordered centrosomes, but also with their positions confined at the desired locations. They thus formed ordered 2-D arrays on the substrate surface. Using this approach, the positions of the microtubule asters on the substrates could be precisely controlled and manipulated. Modification or functionalization of these ordered microtubules with biological or nonbiological materials could also offer a platform for the development of novel, highly sensitive and selective devices to meet a variety of sensing and actuating needs.

### Example 7

### Centrosome Fragment Arrays

I. Centrosome Fragment Preparation

A Sonic Dismembrator (Fisher Scientific) was used to break centrosomes into small fragments. The centrosome sample (2000 uL, 2.5 million/mL) was thawed on ice and diluted to 1 mL with PEM buffer. The sample was then sonicated for 4×5 sec (20 sec total). During the sonication process, samples were immersed into an ice-salt-water-alcohol mixture to avoid being overheated locally by sonication.

In centrosomes, the microtubules are nucleated from the 25 nm γ-TuRC (γ-tubulin ring complex) (Zheng, Y.; Wong, M. L.; Alberts, B.; Mitchison, T. Nature 1995, 378, 578-83) templates on the surface of the centrosomes. When sonicated for short times, centrosomes were fragmented into smaller components, but most γ-TuRCs could still be intact and thus retain their functions. It was observed that centrosome fragments partially lost their functionality when longer sonication time was applied, which may indicate that the structures of γ-TuRCs were also disrupted.

II. Microcontact Printing of Centrosome Fragments

In the previous patterning process for fully constituted centrosomes via a modified microcontact printing (μCp), it was noticed that sometimes centrosomes fragmented into smaller components, if a certain amount of force was applied on the PDMS stamp during the inking step 1.

Following this observation, studies using a general microcontact printing process to pattern centrosome fragments directly onto the glass surfaces were undertaken.

Unlike the patterning of constituted centrosomes, no modification of the printing process was needed for the patterning of centrosome fragments. For the centrosome fragments that are generated in solution by sonication, there are adequate amounts of material so it is not necessary to modify the inking process. Moreover, the amount of centrosome fragments in the solution is high enough so that the solution can be diluted 5-folds from its original concentration for the patterning. The dilution also decreased the viscosity of the solution and thus made the inking process much easier. Different patterns of centrosome fragments, lines or dots, were successfully patterned onto the glass surfaces. On immunofluorescence, no fully constituted centrosomes were identified.

Centrosome fragments were drop-cast onto different PDMS stamps (10 μm dots separated by 10 μm, 10 μm lines separated by 10 μm, a stamp containing mixed patterns of 5 μm dots separated by 30 μm and 5 μm lines separated by 20 μm) and allowed to ink for 2 h at 4° C. The stamps were then briefly dried and brought into contact with the cover slips and printed for approximately 1-2 min. After peeling off the stamps, tubulin solution (made from sea urchin, 0.5 mg/mL, 50 μL) was placed onto each cover slip immediately and incubated at room temperature, 26° C., and allowed to polymerize for 30 min. During the incubation, all the samples were placed in a covered petri-dish containing moistened filter paper at the bottom to prevent evaporation of the tubulin solution.

After tubulin polymerization, all the samples were fixed and subsequently processed for immunofluorescence assay as described herein. Briefly, a 2% (w/v) glutaraldehyde solution in reassembly buffer was used to fix the centrosome fragments and microtubules. After fixing for 15 minutes, all samples were washed with PBS buffer three times and then treated with 10 mg/ml sodium boronhydride for 10 min. After another 3×1 min washing with PBS, samples were blocked with 5% nonfat dry milk in PBS for 20 min. All the samples were then incubated in primary antibody, mouse anti-gamma tubulin and rat anti-alpha tubulin antibodies, for 20 min. Samples were then washed with PBS again and incubated in secondary antibodies, goat anti-mouse IgG and goat anti-rat Alexa Fluor 488, for another 20 min. All the samples were washed with PBS three times and then permanently mounted. All images of the samples were taken with a Zeiss Axioplot epifluorescence microscope equipped with 100×/1.3 NA objective lens and a Coolsnap HQ camera (Photometrics) that was coupled to a Metamorph image processing system (Version 6.2r4, Universal Imaging, Media, Pa.).

Results of these studies indicate that the centrosome fragments retain their functionality in the organization and assembly of microtubules and that fragment templates offer better coverage and patterning flexibilities compared to fully constituted centrosome templates. Furthermore, the patterning process of centrosome fragments is more simple and efficient than that for constituted centrosomes. It was also possible to produce different patterns with different PDMS stamps illustrating the flexibility of the “fragment” system. The amount of organized microtubules could also be controlled by the different concentration of centrosome fragments.

Since the patterned centrosome fragment arrays retain their intrinsic function, they could be used as templates to direct the assembly of microtubules on surfaces with well-defined positions, directions, and polarities and could potentially expand the applications of small organelles, like the centrosome, in the area of bionanotechnologies. These discoveries offer a new system to explore further the assembly of microtubules.

### Example 8

### Directed Assembly of Microtubules on Surfaces by Patterned Centrosome Fragments

Except for very few non-specific binding spots in the background, most microtubules selectively grew from the patterned centrosome fragments. All of the centrosome fragments used for the patterning work were made with a short sonication time (20 sec), so they retained their function very well. Compared with fully constituted centrosomes, centrosome fragments have several advantages in both the patterning and the assembly of microtubules. Firstly, as discussed, the patterning process for the centrosome fragments is simpler than that of fully constituted centrosomes, since no modification is needed for the inking step. Secondly, the centrosome fragments have a wide range of available sizes than that of fully constituted centrosomes (1-2 um). They could thus potentially offer the possibility of patterning of nanosize microtubule organization centers. Thirdly, the solution of centrosome fragments should have much more flexibility in terms of concentration. For fully constituted centrosome patterning, even samples with the maximum concentration that are available would not satisfy the patterning needs for completely covering a useful patterning area. The solutions of centrosome fragments have enough material for the patterning. For example, the present studies produced continuous lines or dots of fragment patterns on surfaces. Moreover, it is also possible to alter the density of microtubules polymerized from the patterns by using different concentrations of centrosome fragments to generate patterns on surfaces. In contract, it is difficult to generate continuous line patterns of fully constituted centrosomes on surfaces, if the same amount of centrosomes is used.

### Example 9

### Production of Continuous Arrays: Connection of Microtubules Aligned from Patterned Centrosomes by Flow Force

There are at least two approaches to achieve continuous arrays of microtubules. Generally, these involve a “lock” to “connect” different microtubules together. In this manner, strong and specific interactions of antibody-antigen and biotin-avidin are applied for “locking” function. Beads coated with anti-tubulin antibodies can be added to the flow cell during or after alignment of microtubules. Each bead could bind to several microtubules through specific antibody-antigen interaction and thus would connect microtubules together. The binding could happen between the microtubules that grow from different centrosomes (along the flow direction) and also between the microtubules that grow from the same centrosome.

A similar approach employs the use of a biotin-avidin interaction instead of antibody-antigen interaction. In this case, microtubules are modified with biotin molecules and when streptavidins are added, the same “locking” function will connect different microtubules together. Once continued arrays of microtubules with unipolarity are generated, they could serve as artificial tracks for unidirectional movement of both motor proteins and different cargos that attached to the motor proteins.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of producing ordered arrays of microtubules comprising; depositing centrosome fragments onto a substrate in an ordered pattern and then contacting said centrosome fragment pattern with tubulin whereby on tubulin polymerization, ordered arrays of microtubules are produced.

2. The method of claim 1 wherein the deposition comprises first contacting a PDMS stamp with a solution of centrosome fragments in an ordered pattern followed by stamping the ordered pattern onto said substrate.

3. The method of claim 2 wherein the substrate is selected from the group consisting of glass, silicon, metal, polymers, composites, ceramic and semiconductors.

4. The method of claim 3 wherein the substrate is glass.

5. The method of claim 2 wherein the substrate is a non-planar surface.

6. An ordered array of microtubules produced by the method of claim 4.

7. A method of directing the growth of microtubles comprising contacting a template which is an ordered array of microtubule organization centers on a substrate with a tubulin solution and applying a field thereby directing the growth of the microtubules along the direction of the field applied.

8. The method of claim 7 wherein the field applied is selected from the group consisting of a liquid flow field, an electrical field, and a magnetic field.

9. The method of claim 7 wherein the microtubule organization centers are selected from the group consisting of centrosomes, centrosome fragments, expressed centrosome proteins, gamma-tubulin ring complexes and microtubule nucleation seeds.

10. The method of claim 7 wherein the template or template substrate is functionalized.

11. The method of claim 10 wherein the functionalization is the attachment of one or more proteins having affinity for microtubules.

12. The method of claim 11 wherein said proteins are selected from the group consisting of kinesin, dynein, microtubule associated proteins (MAPs).

13. The method of claim 12 wherein kinesin, dynein or MAP is further functionalized by the attachment of a metal oxide, metal, semiconductor, or any inorganic or organic moiety.

14. The method of claim 10 wherein the functionalization is the attachment of one or more proteins with no affinity for microtubules.

15. The method of claim 10 wherein the functionalization is the attachment of one or more enzymes.

16. The method of claim 10 wherein the functionalization is the attachment of one or more antibodies.

17. The method of claim 10 wherein the functionalization is the attachment of one or more material tags, wherein said material tags are selected from the group consisting of fluorphores, metal ions and magnetic particles.

18. A system for the unidirectional movement of cargo material carried by microtubule motor proteins comprising a template which is an ordered array of microtubule organization centers, and a directional flow field; wherein said microtubule organization centers are centrosomes or centrosome fragments and wherein the directional flow field is a liquid flow field.

19. The method of claim 1 further comprising a locking system, said locking system being selected from the group consisting of an antigen-antibody system, biotin-streptavidin system and tubulin binding drugs.

20. The method of claim 7 further comprising a locking system, said locking system being selected from the group consisting of an antigen-antibody system, biotin-streptavidin system and tubulin binding drugs.
